(19) United States
(12) Patent Application Publication
(10) Pub. No.: US 2003/0185349 A1
(43) Pub. Date: Oct. 2, 2003

(54) Device for a non-contact measurement of distance at a radiotherapy of the human body

(76) Inventor: Armin Roeckseisen, Adendorf (DE)

Correspondence Address: M.R. FORRESTER, INTOXIMETERS, INC., 1901 LOCUST STREET, ST. LOUIS, MO, 63103

(21) Appl. No.: 10/397,292

(22) Filed: Mar. 26, 2003

(30) Foreign Application Priority Data
Mar. 30, 2002 (DE) 20205049.1

(51) Int. Cl. A61B 6/08

(52) U.S. Cl. 378/206

(57) ABSTRACT

Device for non-contact measurement of a distance to a human body at radiotherapy, comprising a moveable radiation head which directs a treatment beam to the tissue area to be treated, a support provided at the radiation head in a defined distance to the beam, the support is provided for distance measuring means directed to a piercing point, in a measuring position the distance measuring means direct a laser beam to a piercing point of the treatment beam on the skin and measures the distance to the piercing point, and evaluation means computing from the measured distance value the distance of the focus of the treatment beam to the piercing point.

### CROSS-REFERENCE TO RELATED APPLICATIONS

Not Applicable.

### STATEMENT REGARDING FEDERALLY SPONSORED RESEARCH

Not Applicable.

### BACKGROUND OF THE INVENTION

The invention relates to a device for a non-contact measurement of distance at a radiotherapy of the human body, in particular the distance between the focus of the treatment beam to the skin of the patient.

For the radiotherapy it is crucial that a tissue area to be treated, for example a tissue area comprising a tumor, is hit by the treatment beams, and that at the same time the surrounding tissue is contaminated as little as possible. The treatment beam is divergent after the radiation source. In order to limit the treatment area the treatment beam is formed by fixed apertures of metal alloys, so-called blocks, or by leaf apertures, so-called multi leaf-collimators. This allows the tissue area to be ray treated in its varied three-dimensional form to be well scanned by the treatment beam. Arbitrarily formed treatment areas can be ray treated by moving the gantry with the radiation head and additionally moving the support of the patient. It is possible to ray treat the tissue area with a variable radiation power from arbitrary directions. This technique of ray treatment is called “intensity modulated radiotherapy” (IMRT).

However, an important condition for this ray treatment is that the tumor is located in the position expected by the control of the radiotherapy. Concerning this, different positioning and fixation techniques are known. However, already the respiratory movement of the patient may change the position of the tissue area to be ray treated relative to the radiation head and therewith to the focus of the radiation beam.

Linear accelerators for radiotherapy are always equipped with a telemeter. A telemeter is an optical system for measuring the distance between the focus and the skin (SSD). The SSD is measured along the treatment beam for the actual position of the gentry angle. The measurement of the telemeter has to be read out by a human spectator, therefore, the radiotherapy has to be interrupted in order to determine the measurements.

The underlying problem of the invention is to provide a device for non-contact measurement of the distance during radiation, wherein the distance allows with simple means to measure the SSD reliable.

### BRIEF SUMMARY OF THE INVENTION

The device according to the invention has a moveable radiation head directing a treatment beam to an area of tissue to be treated. At the radiation head a support for distance measuring means is provided in a defined distance to the treatment beam. In a measuring position, the distance measuring means direct a laser beam to the piercing point of the treatment beam on the skin, in particular the distance measuring means are directed to the center of the treatment beam. The distance measuring means measure the distance between a defined reference point of the distance measuring means to the piercing point projected onto the skin, according to the principle of laser triangulation. Additionally, the device according to the invention comprises evaluation means which calculate from the measured distance the distance of the focus of the measuring beam to the piercing point. The device according to the invention measures contact-free the distance between skin and focus according to the principle of laser triangulation. In this connection the distance measuring means is positioned off-axis to the treatment beam, and the distance measuring means is directed to the piercing point of the treatment beam on the skin. The distance values are converted with the help of the evaluation means in order to determine the distance along the treatment beam. For the device according to the invention it has proven advantageous that no operator is needed in the treatment room to read out the distance value. Radiotherapy that does not allow a operator in the treatment room can, therefore, be carried out faster since the measurements by an operator need not to read out optically.

The measurement means measure the distance to the laser spot projected onto the skin according to the principle of laser triangulation. For the laser triangulation a laser beam is transmitted. If the laser beam hits an obstacle then a laser spot appears, the position of which is recorded by a light sensitive sensor. Sensor and laser source are provided in a rigidly defined geometrical relation to each other such that form the position and, respectively, the angle under which the laser spot is observed, the distance can be calculated.

In order to bring the distance measuring means in its position the support is provided with an axis of rotation wherein the axis of rotation is perpendicular to the radiation beam. The axis of rotation of the support is aligned such that the laser beam crosses the treatment beam in all positions of the laser beam. The defined position of the axis of rotation relative to the treatment beam allows to calculate the distance along the treatment beam without sensing the angular position of the support.

In a preferred embodiment the measuring principle of the off-axis triangulation is used as protected in U.S. Pat. No. 6,088,106 for the applicant.

In a preferred embodiment, in particular for the use of the intensity modulated radiotherapy, predetermined set points for the distance are provided. The evaluation means switches the radiation source off if deviations of the measured distances to the predetermined set points occur, in the alternative the evaluation means reduce the quality of the radiation source. This step makes sure that only the calculated amount of radiation comes to the predetermined tissue area.

In a preferred continuation of the device according to the invention the measured distance values are used as control input for a position control of the support of the patient and/or for the radiation intensity.

A preferred embodiment of the device according to the invention will be explained in the following by a practical example.

### BRIEF DESCRIPTION OF THE DRAWING

FIG. 1. shows a radiation head 10 directing a divergent treatment beam 12 to a tissue area 14 to be ray treated. The treatment beam 12 is calculated and formed according to the tumor area in a known manner.

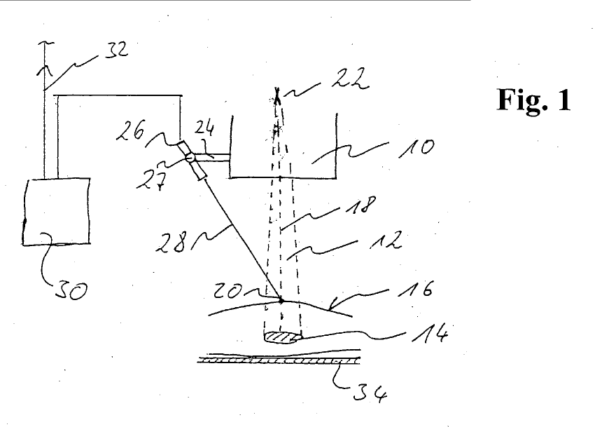

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated.

For the intensity modulated radiotherapy the focus/skin distance (SSD) is a determined value. The radiation beam 12 hits an area of the skin 16. For the intensity modulated radiotherapy the distance along the middle ray 18 is considered. The SSD is determined as the link path of a reference point of the treatment beam 12 to the piercing point 20 of the middle ray. In the FIGURE the assumed focus 22 is drawn by way of example.

Distance measuring means 26 are pivotable arranged at the radiation head 10 by a support 24. The distance measuring means work according to the principle of laser triangulation. For the measurement the laser beam 28 is directed to the piercing point 20. In the piercing point 20 a laser spot is generated that is observed by the distance measuring means 26 under a corresponding angle. If the point 20 is moving up and down the observation angle and the distance along the laser beam 28 change.

Schematically shown evaluation means evaluate the measured distance and calculate from the value the SSD. The SSD is forwarded via a line 32 to a controller.

The distance measuring means 26 are pivotable about an axis perpendicular to the paper plane of the FIGURE. The axis of rotation 27 is aligned such that the laser beam 28 crosses the treatment beam 18 under every angle.

The measured distance value can be used by the controller of the radiation source to modulate the radiation intensity. Also the support for the patient 34 can be arranged in its position depending on the measured distance value.

The above Examples and disclosure are intended to be illustrative and not exhaustive. Thee examples and description will suggest many variations and alternatives to one of ordinary skill in this art. All these alternatives and variations are intended to be included within the scope of the attached claims. Those familiar with the art may recognize other equivalent to the specific embodiment described herein which equivalent are also intended to be encompassed by the claims attached hereto.

1. Device for non-contact measurement of a distance to a human body at radiotherapy, comprising:
a moveable radiation head (10) which directs a treatment beam (12, 18) to the tissue area (14) to be treated,
a support (24) provided at the radiation head (10) in a defined distance to the beam, the support is provided for distance measuring means (26) directed to a piercing point (20), in a measuring position the distance measuring means (26) direct a laser beam (28) to a piercing point (20) of the treatment beam on the skin (16) and measures the distance to the piercing point (20),
and evaluation means computing from the measured distance value the distance of the focus (22) of the treatment beam to the piercing point (20).

2. Device according to claim 1, characterized in that the distance measuring means measure the distance to the laser point according to the principle of laser triangulation.

3. Device according to claim 1, characterized in that the support (26) comprises an axis of rotation (27) perpendicular to the treatment beam (18), and that in every position of the support the laser beam crosses the treatment beam (18).

4. Device according to claim 1, characterized in that the arrangement of laser and sensor comply with the principle of off-axis laser triangulation according to which a con-focal distance measurement is used for the measuring system mounted outside the treatment beam.

5. Device according to claim 1, characterized in that the evaluation means (30) switch the radiation source off when a deviation of the measured distance from a predetermined set value occurs.

6. Device according to claim 5, characterized in that the measured distance value is utilized as control value for a position control of the support of the patient (34).

7. Device according to one of the claim 1, characterized in that the measured distance value is utilized as control parameter for the radiation intensity.
